(19) United States
(12) Patent Application Publication
(10) Pub. No.: US 2012/0129804 A1
(43) Pub. Date: May 24, 2012

(54) METHODS OF REDUCING ABSORPTION OF TRANS FATTY ACIDS USING WATER-INSOLUBLE CELLULOSE DERIVATIVES

(76) Inventors: Kerr W.H. Anderson, Midland, MI (US); David R. Albers, Midland, MI (US); Scott A. Young, Midland, MI (US); Wallace H. Yokoyama, Berkeley, CA (US)

(21) Appl. No.: 13/124,533

(22) PCT Filed: Oct. 16, 2009
(86) PCT No.: PCT/US09/60964

§ 371 Date: Jun. 1, 2011

Related U.S. Application Data
(60) Provisional application No. 61/106,166, filed on Oct. 17, 2008.

(51) Int. Cl. A61K 31/717; A61P 3/00

(52) U.S. Cl. 514/57

(57) ABSTRACT

Provided are food products comprising at least 0.5 g of trans fatty acid per serving and one or more water-insoluble cellulose derivatives in an amount sufficient to reduce absorption of the trans fatty acid by a mammal consuming the food product, methods of ameliorating the harmful effects of trans fatty acids on a mammal that has consumed trans fatty acids, and methods of reducing the amount of trans fatty acids capable of being absorbed by a mammal ingesting a trans fatty acid containing food product.

### CROSS REFERENCE TO RELATED APPLICATIONS

This application claims the benefit of U.S. Provisional Application No. 61/106,166, filed Oct. 17, 2008.

### STATEMENT REGARDING FEDERALLY SPONSORED RESEARCH

This invention was made under a Cooperative Research and Development Agreement with the U.S. Department of Agriculture, number 58-3K95-5-1072.

### FIELD

The present invention relates to mammalian health and nutrition.

### BACKGROUND

Unlike other dietary fats, trans fatty acids are not essential for biological functions, nor are they healthful. To avoid confusion, naturally-occurring trans fat conjugated linoleic acid may offer health benefits, and so “trans fatty acids” typically refers to fatty acids containing one or more trans linkages that are not in a conjugated system.

Trans fatty acids only find their way into food by virtue of their formation in partially-hydrogenated vegetable oils. Trans fatty acids are by-products of partially-hydrogenating vegetable oils, which is done to increase their stability and shelf-life.

Putting aside their fat-based caloric contributions, trans fatty acids in foods have specifically come under strong criticism because trans fatty acids increase low density lipoprotein LDL (bad) cholesterol levels and decrease high density lipoprotein HDL (good) cholesterol levels in humans, increasing the risk of heart disease. Studies also link trans fatty acids to strokes and diabetes type 2. Health authorities recommend avoiding trans fatty acids, and some countries and at least one state in the United States have attempted to ban trans fatty acids in locally prepared foods. The United States Food and Drug Administration (FDA) has taken a different tack, requiring that manufacturers list trans fat on the Nutrition Facts panel of non-institutional foods when present at or over 0.5 g per serving, thus allowing consumers the choice to avoid trans fatty acids. This is not always practical, as partially-hydrogenated vegetable oils (and hence trans fatty acids) are often found in fast foods and snacks, which are highly desirable to even the most informed consumers.

Thus, what is needed are methods of reducing absorption of trans fatty acids by a mammal.

### SUMMARY

In one embodiment, the present invention provides a food product comprising at least 0.5 g of trans fatty acid per serving and one or more water-insoluble cellulose derivatives in an amount sufficient to reduce absorption of the trans fatty acid by a mammal consuming the food product.

In another embodiment, the present invention provides a method of ameliorating the harmful effects of trans fatty acids on a mammal that has consumed trans fatty acids, comprising administering one or more water-insoluble cellulose derivatives to the mammal.

In another embodiment, the present invention provides a method of reducing the amount of trans fatty acids capable of being absorbed by a mammal ingesting a trans fatty acid containing food product, comprising at least one of incorporating one or more water-insoluble cellulose derivatives in the food product, or administering one or more water-insoluble cellulose derivatives before, during or after the consumption of the food product to the mammal.

### DETAILED DESCRIPTION

In one embodiment, the present invention provides a food product comprising at least 0.5 g of trans fatty acid per serving and one or more water-insoluble cellulose derivatives in an amount sufficient to reduce absorption of the trans fatty acid by a mammal consuming the food product.

The term “trans fatty acids” refers to fatty acids containing one or more trans linkages that are not in a conjugated system. As mentioned above, trans fatty acids are an undesirable byproduct of partially-hydrogenated vegetable oils. Elaidic acid is a common trans fatty acid.

Two major avenues for incorporation of trans fatty acids into food involve the addition of margarine or vegetable shortening to the food or frying the food in partially hydrogenated oil. For purposes of this application, the food product may be any comestible typically prepared with partially hydrogenated vegetable oil. Examples of such foods include baked goods (including cookies, crackers, cakes, pies, muffins, donuts, and certain breads such as hamburger buns), deep-fried and pre-fried foods (including doughnuts, french fries, fried chicken, chicken nuggets, fish sticks, and taco shells), snack foods (including potato chips, corn chips, tortilla chips, peanut butter, whipped toppings, instant mashed potatoes, candy, and popcorn), and pre-packaged mixes (including cake mix, pancake mix, biscuit mix, cornbread mix, frosting mix, chocolate drink mix, certain frozen meals, pizza dough, ready to bake bakery products, toaster pastries, waffles, pancakes). Historically, fast food restaurants have been associated with trans fatty acids due to their use of pre-prepared foods and relatively low cost frying oils.

Although prior strategies for avoiding trans fatty acids have been to replace the partially-hydrogenated vegetable oils with costlier alternatives, it is now discovered that incorporation of water-insoluble cellulose derivatives into a mammal's diet has a beneficial effect by reducing the amount of trans fatty acids absorbed by the mammal from ingesting the food. This does not appear to be a “bulk fiber effect,” as water-insoluble cellulose derivatives were found to be more efficacious than microcrystalline cellulose.

Food products of the present invention comprise from 2 to 10 weight percent, more preferably from 2 to 6 weight percent of water-insoluble cellulose derivative, based on the total weight of the food product. The most preferred percentage of water-insoluble cellulose derivative in the food product depends on various factors mainly determined by nutritional and organoleptic considerations.

In one embodiment, the cellulose derivatives which are useful in the present invention are water-insoluble. The term “cellulose derivative” does not include unmodified cellulose itself. The term “water-insoluble” as used herein means that the cellulose derivative has a solubility in water of less than 2 grams, preferably less than 1 gram, in 100 grams of distilled water at 25° C. and 1 atmosphere. Preferred water-insoluble cellulose ethers are ethyl cellulose, propyl cellulose, or butyl cellulose. Other useful water insoluble cellulose derivatives are cellulose derivatives which have been chemically, preferably hydrophobically, modified to provide water insolubility. Chemical modification can be achieved with hydrophobic long chain branched or non-branched alkyl, arylalkyl or alkylaryl groups. “Long chain” typically means at least 5, more typically at least 10, particularly at least 12 carbon atoms. Other types of water-insoluble cellulose are crosslinked cellulose, when various crosslinking agents are used. Chemically modified, including the hydrophobically modified, water-insoluble cellulose derivatives are known in the art. They are useful provided that they have a solubility in water of less than 2 grams, preferably less than 1 gram, in 100 grams of distilled water at 25° C. and 1 atmosphere pressure. The most preferred cellulose derivative is ethyl cellulose. The ethyl cellulose preferably has an ethoxyl substitution of from 40 to 55 percent, more preferably from 43 to 53 percent, most preferably from 44 to 51 percent. The percent ethoxyl substitution is based on the weight of the substituted product and determined according to a Zeisel gas chromatographic technique as described in ASTM D4794-94 (2003). The molecular weight of the ethyl cellulose is expressed as the viscosity of a 5 weight percent solution of the ethyl cellulose measured at 25° C. in a mixture of 80 volume percent toluene and 20 volume percent ethanol. The ethyl cellulose concentration is based on the total weight of toluene, ethanol and ethyl cellulose. The viscosity is measured using Ubbelohde tubes as outlined in ASTM D914-00 and as further described in ASTM D446-04, which is referenced in ASTM D914-00. The ethyl cellulose generally has a viscosity of up to 400 mPa·s, preferably up to 300 mPa·s, more preferably up to 100 mPa·s, measured as described above. The preferred ethyl celluloses are premium grades ETHOCEL ethyl cellulose which are commercially available from The Dow Chemical Company of Midland, Mich. Combinations of two or more water-insoluble cellulose derivatives are also useful.

Preferably the water-insoluble cellulose derivative has an average particle size of less than 0.1 millimeter, more preferably less than 0.05 millimeter, most preferably less than 0.02 millimeter.

In yet another embodiment, the present invention provides a method of ameliorating the harmful effects of trans fatty acids on a mammal that has consumed trans fatty acids, comprising administering one or more water-insoluble cellulose derivatives to the mammal.

In yet another embodiment, the present invention provides a method of reducing the amount of trans fatty acids capable of being absorbed by a mammal ingesting a trans fatty acid containing food product, comprising at least one of incorporating one or more water-insoluble cellulose derivatives in the food product; or administering one or more water-insoluble cellulose derivatives before, during or after the consumption of the food product to the mammal.

The presence of one or more water-insoluble cellulose derivatives in the food product or the administration of one or more water-insoluble cellulose derivatives in combination with the food product is particularly effective. The water-insoluble cellulose derivatives are preferably incorporated in such amount in the food product or are administered in such amounts before, during or after the consumption of the food product such that the daily dose of water-insoluble cellulose derivative is generally in the range of 20 to 700 milligrams of water-insoluble cellulose derivative per kilogram of mammal body weight per day. Preferably about 2 to 30, more preferably about 3 to 25 g of water-insoluble cellulose derivative are ingested by a large mammal such as a human. The most preferred amount of water-insoluble cellulose derivative depends on various factors, such as the fat content of the diet.

When the water-insoluble cellulose derivative is administered separately from the food product, it can, for example be administered in the form of powdered, reverse-enteric coated or micro-encapsulated cellulose ether suspended in a flavored drink formulation, as tablets, capsules, sachets or caplets. When the water-insoluble cellulose derivative is administered separately from the food product, it should be consumed in an appropriate timeframe with the food product, preferably within about 30 minutes before or after consumption of the food product, more preferably within about 15 minutes before or after consumption of the food product.

### EXAMPLES

The following examples are for illustrative purposes only and are not intended to limit the scope of the present invention. All percentages are by weight unless otherwise specified.

### Example 1

Male Syrian Golden hamsters with a starting body weight of between 80-90 grams (LVG strain, Charles River Laboratory, Willmington, Mass.) were fed standardized laboratory food (lab “chow”) for seven days to acclimate.

The acclimated hamsters were then assigned to one of the six groups in TABLE 1:

TABLE 1

| | Supersize | | Pound Cake | | Pizza | |
|---|---|---|---|---|---|---|
| | Treat- | | Treat- | | Treat- | |
| Component | ment | Control | ment | Control | ment | Control |
| Supersize | 1700 | 1700 | — | — | — | — |
| Pound Cake | — | — | 480 | 480 | — | — |
| Pizza | — | — | — | — | 1250 | 1250 |
| Vitamin Mix | 17 | 17 | 5 | 5 | 13 | 13 |
| Mineral Mix | 60 | 60 | 17 | 17 | 46 | 46 |
| Corn oil | — | — | 10 | 10 | — | — |
| Casein | — | — | 58 | 58 | — | — |
| EC | 71 | — | 20 | — | 52 | — |
| MCC | — | 71 | — | 20 | — | 52 |

Components are listed in grams, with further details below:

Supersize—meals of hamburgers/french fries that were freeze dried and powdered. Energy intake was about 33.62%, and the trans fats content about 1.3%.

Pound Cake—freeze dried and powdered pound cake. Energy intake was about 32.8%, and the trans fats content about 0.7%.

Pizza—freeze dried and powdered pizza. Energy intake was about 26.5%, and the trans fats content about 0.3%.

Vitamin Mix—1.0 g/Kg Vitamin A Palmitate (500,000 IU/g), 0.6 g/Kg Vitamin D3 (400,000 IU/g), 10.0 g/Kg Vitamin E Acetate (500 IU/g), 10.0 g/Kg Inositol, 0.4 g/Kg Menadione Sodium Bisulfate, 9.0 g/Kg Niacin, 1.5 g/Kg Riboflavin, 2.0 g/Kg Thiamine HCl, 0.7 g/Kg Pyridoxine HCl, 4.0 g/Kg Calcium Pantothenate, 0.06 g/Kg Biotin, 0.2 g/Kg Folic Acid, 1.0 g/Kg Vitamin B12 (0.1%), and 959.54 g/Kg Sucrose. [From Recommendations by NRC Nutrient Requirements of Laboratory Animals, 3^{rd} Revised Edition, 1978. Dyets, Inc., Dr. H. L. Yowell]

Mineral Mix—388.2 g/Kg potassium phosphate, dibasic, 85.6 g/Kg calcium phosphate, dibasic, 363.9 g/Kg calcium carbonate, 109.0 g/Kg sodium choloride, 28.56 g/Kg magnesium oxide, 22.94 g/Kg ferric citrate, U.S.P., 0.06 g/Kg cobaltous carbonate, 0.29 g/Kg cupric carbonate, 0.01 g/Kg sodium fluoride, 0.06 g/Kg potassium iodide, 0.22 g/Kg manganese carbonate, 1.11 g/Kg zinc carbonate, 0.04 g/Kg chromium acetate, 0.01 g/Kg sodium selenite. [From Recommendations by NRC Nutrient Requirements of Laboratory Animals, 3^{rd} Revised Edition, 1978. Dyets, Inc., Dr. H. L. Yowell]

EC—Standard 10F Premium FP grade ethyl cellulose (EC) from The Dow Chemical Company. When present, the dosage was approximately 4% (wt/wt).

MCC—microcrystalline cellulose (MCC). When present, the dosage was approximately 4% (wt/wt).

The study was approved by the Animal Care and Use Committee, Western Regional Research Center, USDA, Albany, Calif., and all guidelines for the care and use of laboratory animals were followed. The animals were fed the diet from TABLE 1 assigned to them for 20 days, with collection of feces at 10 days and 20 days. The feces were freeze-dried at each collection time.

For the fecal fat content analyses, feces were ground for 10 minutes on a SPEX 8000 or 80000 mixer mill using two tungsten carbide balls in a tungsten carbide cylinder. About 0.15 g of ground fecal matter was weighed into a DIONEX 11 mL Accelerated Solvent Extractor (ASE) cell and combined with about 3.5 g diatomaceous earth (sand) with brief stirring. 100 μL of a 500 μg/mL solution of glyceryl trierucate in tetrahydrofuran (THF) was added to the cell, and the mixture was sandwiched between two cellulose filters.

The cell contents were extracted with an extraction solvent containing 600 mL of hexane, 400 mL 2-propanol, and 20 mL acetic acid on a DIONEX ASE system with the following conditions: Preheat 1 min, pressure 2175 psi, heat 5 min, Temp 60° C., Static 10 min, flush 60%, purge 120 sec, cycle=2. This resulted in 20 mL of sample extract collected.

After mixing, 9.0 mL of extract was placed in a tared 16×125 mm screw top culture tube. The extract was blown to dryness in a 45° C. water bath with a nitrogen purge. A 4 mL aliquot of acetonitrile was added and the mixture was blown to dryness again.

The samples were derivatized using the following procedure:
1. A 300 μL aliquot of 0.5N NaOH in MeOH (methanol) was added to each sample in the culture tube.
2. Tubes were capped, vortexed, and placed in a heating block at 100° C. for 5 min.
3. Samples were allowed to cool about 1 min.
4. Samples were uncapped and 350 μL of 14% boron trifluoride in MeOH was added.
5. Samples were capped, vortexed, and placed in a heating block at 100° C. for 5 min.
6. Samples were allowed to cool about 1 min.
7. Vials were uncapped and 2 mL heptane (0.200 mg/mL nonadecane in heptane) was added.
8. Vials were recapped, vortexed, and placed back in heating block at 100° C. for 5 min.
9. Samples were allowed to cool for about 1 min.
10. Vials were uncapped and 1 mL salt saturated H₂O solution was added.
11. They were recapped and placed on rocker for 5 min.
12. They were then centrifuged at 1500 rpm for 10 min.
13. Using Pasteur pipettes, about 1 mL of organic (top) layer was transferred into gas chromatography (GC) vials.
   The derivatized samples were analyzed by gas chromatography with the following conditions:

Chromatograph: Agilent 6890 Series GC
Column: 60 m×0.25 mm (L×ID), 0.25 μm df,
Detector FID (Flame Ionization Detector)

Temperatures:

Oven: 200° C. (5 min), to 250° C. at 5° C./min with 5 min. final hold time

Injector: 250° C.

Detector: 260° C.

Carrier: 3 mL/min at 200° C. (62.5 psi)

Split: 25 mL/min

Make-Up: Helium 27 mL/min

Air: 400 mL/min

Hydrogen: 30 mL/min

Sample Size: 2 μL

Data System: EZChrom Elite Version 3.2.1

For calibration, a spiking standard was prepared to contain 0.200 mg/mL nonadecane and 0.10 mg/mL methyl erucate in heptane. A calibration solution was prepared by weighing out 10 mg of NuCheck Standard 1A into a 1.0 mL volumetric flask. To this flask 110 μL of the spiking standard was added. The flask was diluted to volume with heptane containing 0.200 mg/mL nonadecane. NuCheck Standard 1A contained 20% each of methyl palmitate (C16:0), methyl stearate (C18:0), methyl oleate (C18:1), methyl linoleate (C18:2) and methyl linolenate (C18:3). Thus the calibration solution contained 2000 μg/mL of these five components along with 200 μg/mL nonadecane and 11 μg/mL methyl erucate.

Trans fatty acids were determined by summing the monounsaturated trans fatty acids C16:1, C18:1, C18:2 and C20:1. Trans fatty acids were identified by spiking experiments and by elution order provided in literature from the GC column manufacturer. The results are listed in TABLE 2:

TABLE 2

| | Supersize | | Pound Cake | | Pizza | |
|---|---|---|---|---|---|---|
| | Day 10 | Day 20 | Day 10 | Day 20 | Day 10 | Day 20 |
| Treatment | 9* | 8.6 | 9 | 6 | 2* | 1.5* |
| Control | 2 | 4.2 | 6 | 5 | 0.4 | 0.6 |

Results are reported in mg/g. The treatment group (fed with 4% EC) had a significant increase in the excretion of trans fatty acids over the control group. Thus, EC facilitated the reduction of trans fatty acids being absorbed by an animal body. The data noted with an asterisk were significant at the 95% confidence interval.

### Example 2

Male Syrian Golden hamsters with a starting body weight of between 80-90 grams (LVG strain, Charles River Laboratory, Willmington, Mass.) were fed standardized laboratory food (lab “chow”) for seven days to acclimate.

The acclimated hamsters were then assigned one of the groups described in TABLE 3:

TABLE 3

| | Bologna | | Cheese | | Potato Chip | |
|---|---|---|---|---|---|---|
| | Treat- | | Treat- | | Treat- | |
| Component | ment | Control | ment | Control | ment | Control |
| Bologna | 33 | 33 | — | — | — | — |
| Cheese | — | — | 35 | 35 | — | — |
| Potato Chip | — | — | — | — | 56 | 56 |
| Vitamin Mix | 1 | 1 | 1 | 1 | 1 | 1 |
| Mineral Mix | 3.5 | 3.5 | 3.5 | 3.5 | 3.5 | 3.5 |
| Corn oil | 2.5 | 2.5 | 2.5 | 2.5 | 2.5 | 2.5 |
| Casein | 10 | 10 | 8 | 8 | 12 | 12 |
| DL-methionine | 0.3 | 0.3 | 0.3 | 0.3 | 0.3 | 0.3 |
| Cornstarch | 45.4 | 45.4 | 45.4 | 45.4 | 20.4 | 20.4 |
| Choline | 0.3 | 0.3 | 0.3 | 0.3 | 0.3 | 0.3 |
| Biturate | | | | | | |
| EC | 4 | — | 4 | — | 4 | — |
| MCC | — | 4 | — | 4 | — | 4 |

Components are listed in grams, with further details below:

Bologna—bologna that was freeze dried and powdered, having a trans fat content of about 1.0%.

Cheese—cheese that was freeze dried and powdered, having a trans fat content of about 0.4%.

Potato Chip—potato chips that were freeze dried and powdered, having a trans fat content of about 0.3%.

Vitamin Mix—1.0 g/Kg Vitamin A Palmitate (500,000 IU/g), 0.6 g/Kg Vitamin D3 (400,000 IU/g), 10.0 g/Kg Vitamin E Acetate (500 IU/g), 10.0 g/Kg Inositol, 0.4 g/Kg Menadione Sodium Bisulfate, 9.0 g/Kg Niacin, 1.5 g/Kg Riboflavin, 2.0 g/Kg Thiamine HCl, 0.7 g/Kg Pyridoxine HCl, 4.0 g/Kg Calcium Pantothenate, 0.06 g/Kg Biotin, 0.2 g/Kg Folic Acid, 1.0 g/Kg Vitamin B12 (0.1%), and 959.54 g/Kg Sucrose. [From Recommendations by NRC Nutrient Requirements of Laboratory Animals, 3^{rd} Revised Edition, 1978. Dyets, Inc., Dr. H. L. Yowell]

Mineral Mix—388.2 g/Kg potassium phosphate, dibasic, 85.6 g/Kg calcium phosphate, dibasic, 363.9 g/Kg calcium carbonate, 109.0 g/Kg sodium choloride, 28.56 g/Kg magnesium oxide, 22.94 g/Kg ferric citrate, U.S.P., 0.06 g/Kg cobaltous carbonate, 0.29 g/Kg cupric carbonate, 0.01 g/Kg sodium fluoride, 0.06 g/Kg potassium iodide, 0.22 g/Kg manganese carbonate, 1.11 g/Kg zinc carbonate, 0.04 g/Kg chromium acetate, 0.01 g/Kg sodium selenite. [From Recommendations by NRC Nutrient Requirements of Laboratory Animals, 3^{rd} Revised Edition, 1978. Dyets, Inc., Dr. H. L. Yowell]

EC—Standard 10F Premium FP grade ethyl cellulose (EC) from The Dow Chemical Company. When present, the dosage was approximately 4% (wt/wt).

MCC—microcrystalline cellulose (MCC). When present, the dosage was approximately 4% (wt/wt).

The study was approved by the Animal Care and Use Committee, Western Regional Research Center, USDA, Albany, Calif., and all guidelines for the care and use of laboratory animals were followed. The animals were fed the diet from TABLE 3 assigned to them, with collection of feces at 6 days.

Trans fatty acids content in the feces was analyzed as described in Example 1, and the results are listed in TABLE 4:

TABLE 4

| | Bologna | Cheese | Potato Chip |
|---|---|---|---|
| Treatment | 12 | 4.9* | 2.6 |
| Control | 13 | 2.8 | 1.5 |

Results are reported in mg/g. The treatment group (fed with 4% EC) had a significant increase in the excretion of trans fatty acids over the control group except with respect to the bologna diet. Thus, EC facilitated the reduction of trans fatty acids being absorbed by an animal body. The data noted with an asterisk were significant at the 95% confidence interval.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A food product comprising:
at least 0.5 g of trans fatty acid per serving; and
one or more water-insoluble cellulose derivatives in an amount sufficient to reduce absorption of the trans fatty acid by a mammal consuming the food product.

2. The food product of claim 1, wherein the water-insoluble cellulose derivative is ethyl cellulose.

3. The food product of claim 1, wherein the water-insoluble cellulose derivative is present from 2 to 10 weight percent, more preferably from 2 to 6 weight percent, based on the total weight of the food product.

4. The food product of claim 1, wherein the food product is prepared with partially hydrogenated vegetable oil.

5. A method of ameliorating the harmful effects of trans fatty acids on a mammal that has consumed trans fatty acids, comprising:
administering one or more water-insoluble cellulose derivatives to the mammal.

6. The method of claim 5, wherein the water-insoluble cellulose derivative is ethyl cellulose.

7. The method of claim 5, wherein the mammal is a human, and the water-insoluble cellulose derivative is administered in an amount from about 2 to 30 g, more preferably about 3 to 25 g.

8. A method of reducing the amount of trans fatty acids capable of being absorbed by a mammal ingesting a trans fatty acid containing food product, comprising the steps of:
incorporating one or more water-insoluble cellulose derivatives in the food product; or
administering one or more water-insoluble cellulose derivatives before, during or after the consumption of the food product to the mammal.

9. The method of claim 8, wherein the water-insoluble cellulose derivative is ethyl cellulose.

10. The method of claim 8, wherein the water-insoluble cellulose derivative is present from 2 to 10 weight percent, more preferably from 2 to 6 weight percent, based on the total weight of the food product.

11. The method of claim 8, wherein the food product is prepared with partially hydrogenated vegetable oil.

12. The method of claim 8, wherein the mammal is a human, and the water-insoluble cellulose derivative is administered in an amount from about 2 to 30 g, more preferably about 3 to 25 g.
